(19) United States
(12) Patent Application Publication
(10) Pub. No.: US 2007/0250341 A1
(43) Pub. Date: Oct. 25, 2007

(54) Method for providing a consumer with information regarding commercial prescription availability and cost

(75) Inventors: Frederick Howe, La Jolla, CA (US); Dale R. Brown, Poway, CA (US); John Tauber, San Diego, CA (US)

Correspondence Address: GORDON & REES LLP, 101 WEST BROADWAY, SUITE 1600, SAN DIEGO, CA, 92101, US

(73) Assignee: Medimpact Healthcare Systems, Inc.

(21) Appl. No.: 11/408,823

(22) Filed: Apr. 20, 2006

(51) Int. Cl. G06Q 10/00; A61B 5/00

(52) U.S. Cl. 705/2; 600/300

(57) ABSTRACT

A method is disclosed whereby consumers enrolled in subscribing prescription benefit plans can easily obtain information over the Internet regarding costs and availability of their prescription medications at local and mail-order participating pharmacies of their choice, based on the prescription benefit plans of which they are members. Data on medication dosages, quantities and net costs at a plurality of pharmacies are compiled by the method and presented on-line so that each member of a plan can select the combination of cost and convenience for prescription fulfillment that he or she prefers. The method then allows the user to select prescription fulfillment directly from the selected pharmacy on-line. Other information,.such as general medical, medication and health information can also be made available. Patients, pharmacies, PBMs and plan payers all benefit.

### FIELD OF THE INVENTION

The present invention relates generally to the field of providing information about filling prescriptions for consumers. More particularly it relates to stimulating competition among pharmacies for filling of patients' prescriptions and enabling patients to select those pharmacies for prescription options which best meet their needs and preferences.

### BACKGROUND OF THE INVENTION

Consumer-directed health plans increase consumer engagement in health care decision-making and health management, and improved care for those with chronic diseases. But the biggest stumbling block right now is the lack of consumer-friendly decision support tools, especially information on prices. Therefore, what is needed is a consumer web portal that creates an open marketplace where pharmacies compete for consumer business by providing choices of pharmacies and locations, choices of brand and generic drugs, choices of co-payment and out of pocket pricing options, choices of fulfillment options, and relevant safety, health and wellness information.

Many employees and members (“consumers”) of health maintenance organizations, employer groups and government entities have their purchases of personal prescription medications subsidized by payments to pharmacies through prescription benefit plans (“plans”) offered by those health maintenance organizations, employer groups and government entities. For the purposes of this invention, “plans” includes the newly enacted Part D of Medicare. Under such plans, a consumer receives a prescription for a medication from his or her physician and submits it to a pharmacy to be filled. The pharmacy checks to see that the consumer is a member of a plan with which the pharmacy has a contract and that the medication and dosage prescribed are within the approved scope of the plan contract. Upon verification of these requirements, the pharmacy dispenses the medication to the consumer. The consumer pays the pharmacy a “copay” amount, less than the normal cost of the medication. The pharmacy receives the balance of the payment for the medication and its dispensing services from the prescription benefit plan, which is managed by a “prescription benefit manager” (“PBM”) with whom the health maintenance organization, employer group or government entity (“payer”) has contracted to manage the plan. The PBM invoices the payer (i.e., the PBM's customer) for the consumer's transaction, along with a charge for its contracted fee, and from the funds paid by the payer the PBM pays the pharmacy's balance due.

Medication usage is commonly differentiated between acute care usage, which is short term (30 days or less) administration to treat immediate illnesses or conditions, and maintenance usage, which is long term (more than 30 days) treatment of chronic illnesses or conditions such as hypertension, high cholesterol levels, arthritis and the like. Maintenance medication dispensing and usage represents a major health care cost (on the order of 75% of prescription costs for many plans, especially due to the aging of the American population) and therefore control of maintenance prescription costs is a principal function of the prescription benefit plans. Dispensing pharmacies are normally of two types: retail pharmacies (which are local neighborhood businesses where the consumer appears in person, can meet with a pharmacist, orders his/her medication and can usually leave a few minutes later with the dispensed medication in hand) and mail order pharmacies (which are large facilities, usually not open to individual consumers' personal visits, but from which a consumer's medication order received by mail or through the Internet is subsequently filled and dispensed to the consumer via mail or courier service). It is normally recognized by the industry that acute care prescriptions are dispensed primarily by retail pharmacies, since the consumer frequently needs the medication immediately and cannot accept the multi-day delay inherent in submitting and dispensing prescription medications from the mail order pharmacies.

Because of the expense of medications, it is in the interest of PBMs, payers and patients for patients to be able to obtain their prescribed medications at the lowest costs consistent with medication availability and convenience to the patient. Numerous factors enter into the determination of what is optimum for each individual patient when obtaining his or her medications. A patient may consider, for instance, how far he or she must travel to different local retail pharmacies, the time difference involved in patronizing a local retail pharmacy as compared to obtaining medications from a mail-order pharmacy, and/or whether a particular pharmacy maintains a supply of a specific medication on hand or must special order it. However, commonly the determining consideration of the patient is the cost of the medication at each of the various pharmacies which meet the patient's other considerations of convenience. A patient is often likely to accept the need for longer travel and time to a more distant pharmacy if that pharmacy's price for the medication is lower than the price at a pharmacy which is more conveniently located. Similarly, a patient is often likely to accept the time delay in obtaining a medication by shipment from a mail-order pharmacy if that pharmacy's price is lower than the price at a local pharmacy where the patient could obtain the medication more quickly.

However, frequently these benefits to the patient, the payer and the PBM cannot be obtained because the patient and the payer do not know and cannot readily determine what prices are being charged for a particular medication at different pharmacies. Theoretically a patient can shop several different pharmacies, either by traveling to each or telephoning each, to determine which provides the best combination of price and convenience. However, in practice that is not really feasible, since few patients can spend the time visiting or communicating with numbers of different pharmacies, nor can the pharmacists or staff at the various pharmacies easily take time from their work to respond to such “shopping” inquiries. Consequently, a patient usually ends up simply obtaining his or her medications from a pharmacy recommended by the PBM or payer of the plan that he or she is on, or by referring to some self-interested source such as a pharmacy's newspaper advertisement, without having means to recognize that another pharmacy (or perhaps several other pharmacies) convenient to the patient has the same medication available at a substantially lower cost.

It is therefore evident that patients (and thus also payers and PBMs) would greatly benefit by being able to obtain, conveniently and from a single easily available source, comparative price information for a given medication from a number of pharmacies to which the patient has reasonable access. The patient could then access such source, examine the cost of the prescribed medication under his or her particular prescription plan at each of the listed pharmacies, and then make an educated selection of which pharmacy to patronize from that information. For such a program to work, of course, it must also have input from the payer and the PBM, since the price to a particular patient will be determined by the limitations defined in the patient's plan. The patient would then benefit by getting the lowest cost consistent with his or her other considerations, the payer would benefit by having the patient encouraged to patronize the pharmacies which meet the payer's cost and funding expectations for the plan, and the PBM would benefit by being better able to administer the plan by focusing on the various pharmacies which consistently provide services whose cost/convenience factors are most attractive to patients.

### SUMMARY OF THE INVENTION

The method of the present invention is a method for presenting to prescription drug patients, on essentially a current basis, detailed information about the availability and costs of their prescription medications at local and mail-order pharmacies, allowing them to select the specific combination of cost and convenience that they determine to best meet their needs and wants, and then arranging for those prescription selections to be filled as they instruct. The method involves the following steps. A database is provided which includes information about various prescription medications including forms and dosages for such medications and participating pharmacies which dispense those medications including the locations of such pharmacies and their retail prices for such medications. For certain drugs in certain therapeutic classes, an equivalency table will be constructed that suggests a particular drug with a particular dosage may be substituted for another drug with another dosage. Information from prescription benefit plans which subscribe to the database as to the benefit amounts paid by the plans on behalf of their members for those medications is included in the database. The software operating the database calculates and displays the net payment chargeable to a prescription patient when filing a prescription for a specific medication for each pharmacy reasonably available to the patient, based on the pharmacy's retail price and the benefit payment paid by the patients prescription benefit plan. By accessing the database via the Internet the patient can identify the combination of pharmacy and net medication cost that the patient deems most satisfactory to him or her and exercise his or her personal choice of which pharmacy to patronize for that medication.

In a principal embodiment of the method, the relevant information is presented to a patient on an Internet web site in tabular form, based on personal information initially submitted by the patient to identify the patient's location (usually residence address), the specific medications and their dosages, forms and quantities sought and the prescription plan of which the patient is a member. The tabular presentation commonly shows each medication with its dosages, forms and quantities that are clinically or therapeutically equivalent available for each participating pharmacy within a selected geographical range from the patient's location as well as mail-order pharmacies which the patient may elect to patronize. For each combination of pharmacy and medication quantity, the presentation shows the payment which the patient's prescription plan will pay to the pharmacy on the patient's behalf and net cost to the patient after the plan's payment is credited against the pharmacy's retail price. Means are provided on-line for the patient to make his or her selection of the pharmacy and drug based on the combination of price and convenience which most nearly meets the patient's own preference.

The method allows the patient free choice among what will usually be a significant number of different combinations of pharmacies and prices. While each prescription benefit plan will have its own schedule of allowed payments for medications available under that plan, different retail prices charged by different pharmacies may result in a variety of prices available to a patient. If the patient chooses the lowest cost option based on choices of drug, pharmacy and fulfillment, the patient pays zero copay. Since the method is intended to maximize the patient's choices, however, that net zero option is not dictated to the patient by the method. Rather the patient still has the option of selecting a different option which has a non-zero net cost to the patient, thus giving the patient the choice of paying a net cost out of his or her own pocket in response to the patient's own criteria for selection. For instance, the net zero cost option may only be available at a pharmacy which is located farther from the patient's location than the patient wishes to travel to obtain the prescription. The method thus gives the patient the ability to obtain the prescription more conveniently at a closer pharmacy by electing to pay a non-zero net cost on his or her own.

Optionally the method may include numerous additional features of value to the patients, pharmacies, plan administrators and plan payers. The database will normally also include means for recording a patient's medication history and will be able to recognize combinations of medications for which there are contraindications and then present that information to the patient, encouraging the patient to consult with his or her physician to determine whether one or another medication in a contraindicated combination should be changed. The database will normally also include general information about various medications, so that a patient can research medications for a medical condition prior to or as part of consulting a physician about the condition, so that the patient can discuss his or her situation more intelligently with the physician. In a preferred embodiment such general information (which does not include pharmacy or prescription plan pricing) will be available to the general public and not limited only to members of subscribing prescription benefit plans.

The method also preferably provides means for collecting information about usage, pharmacy patronage, and medication cost selection which can be analyzed by the participating pharmacies and prescription benefit plans. Such analyses can allow the plans and pharmacies to determine, for instance, whether patients are preferentially selecting the brand version or generic version of a medication, if both are available, or preferentially patronizing local retail or mail-order pharmacies. Such reports may also suggest to a pharmacy that a change in its retail pricing may increase its business, or to a prescription plan that a change in its benefits payment schedule may result in increasing its membership, or to a plan payer organization that it should consider changing to a different plan to provide greater benefits to its employees or members.

The method of this invention does not determine or dictate retail prices or prescription plan benefit payment schedules, nor does it artificially limit or dictate patients' patronage options. Rather it compiles and presents to the patient, in a unique and highly effective and informative manner, the information that the patient needs to be able to obtain the medications that his or her physician has prescribed at the cost and with the degree of convenience that the patient personally chooses. It does not, however, permit patients to change their prescriptions without the consent of their doctors. Rather it highlights the different options for the patients and encourages patient-physician interaction. Patients are therefore pleased because they have obtained their prescriptions based on their own selection criteria, payers are pleased because their employees/members are satisfied with the prescription benefit plans they provide, pharmacies are pleased because they have the opportunity to reach customers (patients) that would otherwise be unlikely to be aware of their services, and prescription benefit plans (and their customers, the payers) are pleased because the information presented to the patients encourages them to select the most cost-effective options as determined by the plans.

Other embodiments, features, examples and benefits of the method of this invention will be disclosed or evident from the portion of this specification which follows below.

### DETAILED DESCRIPTION OF THE INVENTION

As noted above, the invention herein is a method for presenting to prescription drug patients, on essentially a current basis, detailed information about the availability and costs of their prescription medications at local and mail-order pharmacies, allowing them to select the specific combination of cost and convenience that they determine to best meet their needs and wants, and then arranging for those prescription selections to be filled as they instruct. Involved in this invention are four entities; the patients, the pharmacies, the payers of the prescription plans in which the patients are enrolled, and the prescription benefits managers for those plans. As will be disclosed below, all four of these entities benefit in manners not available from prior art methods of providing prescriptions to patients.

Considering first the patients, the benefits obtained are that the patients, having identified themselves, their locations and their medications to system, are then quickly provided with a concise and easily understood list of pharmacies which are either conveniently located close to the patients' locations or provide mail-order services, carry the desired medications, and are recognized as eligible prescription providers by the patients' prescription benefits plans. (For the purpose of discussion and examples in this specification, patients will be exemplified by an adult female whose suffers from hypertension and has been prescribed the branded antihypertensive medication Prinzide® 25, [Merck Corporation] which is a combination of 25 mg of hydrochlorothiazide [a diuretic] and 20 mg of lisinopril [an angiotensin-converting enzyme inhibitor], and an adult male who suffers from hyperlipidemia with elevated serum cholesterol and low-density lipoprotein levels and has been prescribed the branded antihyperlipidemic medication Zocor® [simvastatin: Merck Corporation] in a dosage of 20 mg.) Patients can then easily review the list of pharmacies and prices, determine which has in their view the best combination of price and convenience, and order their physician-approved prescriptions, usually either via mail order or by direct contact with the selected pharmacy.

For payers and the PBMs, the benefits are that the employees or members of the payer organizations (i.e., the patients) are pleased with the convenience and choice provided by the system of the invention and their satisfaction is reflected to the payers and the PBMs managing the plans, not only by express statements but also by a reduction in what would otherwise be the expected level of complaints arising from the patients.

Finally, for the pharmacies the benefits include increased and continued business from satisfied customers, particularly since the customers (patents) feel empowered by the system to be able to select the pharmacies to patronize on the basis of their own criteria, and not have a specific pharmacy choice forced on them by their plan. It is recognized that a person who is able to choose which provider to patronize will initially view that provider more favorably than a person who has been directed to patronize a provider selected by someone else, and also that the person who freely chose the provider is more likely to remain with that provide without complaints than is the person who had no choice in the matter. For pharmacies also, the system provides a means by which they can reach potential customers that they otherwise would not have had contact with, and perhaps would not even have been aware of. This gives the pharmacies the opportunity to increase their business substantially at a modest cost.

The system is structured around an Internet site on which resides software embodying the system/method of this invention. Pharmacies in numerous locations pay the operator of the Internet site a fee to be included in the system's database. Data in the database include identification of each subscribing pharmacy and its physical location (for local retail pharmacies) or mailing address (for mail-order pharmacies), a list of the medications which the pharmacy carries in stock or can readily obtain, and the pharmacy's current retail price for the various standard dosages and forms (liquid, tablet, etc.) of each medication. The system software allows for sorting of data in response to patient inquiries by medication, pharmacy location, medication cost, fulfillment choices (retail 30-day, retail 90-day, mail order) and availability. Prescription plans through their PBMs and with payer approval also subscribe to the system of the invention, and the database data commonly include identification of the medications that each plan approves, the level of approval required by the plan for a particular medication, the portion of the cost of each medication that the plan is willing to pay, and the co-pay amount (if any) for each medication that the plan requires its participants to bear.

In addition to the basic prescription and pharmacy information that the database includes, the system also provides means for patients accessing the site to identify appropriate medications for their conditions by medication name, medication type, symptoms addressed by a medication, and other entry criteria for the database which may be useful to the patients. Thus, our exemplary female hypertensive could identify numerous hypertension medications on the Internet site and learn of their properties from information about the medications included in the database, including adverse effects whose applicability to her health might not have previously been considered. The information thus obtained by the patient in turn would allow her to discuss her medication regimen more intelligently with her physician. Further, the system does or can provide general public information about medication-related issues; provide warning of newly recognized medication problems; identify symptoms, diagnoses and treatments of various diseases; or educate as to many other health-related topics. It is also contemplated that the database will include relevant information for patients about their local hospitals, including information about types of hospital services provided, particular expertises that a hospital may have related to a specific disease, group of diseases or physical condition or dysfunction, as well as information on treatment outcomes, costs and safety in various hospitals. The general and educational types of information mentioned above will normally be available generally to visitors to the Internet site, regardless of whether they are members of a subscribing prescription benefit plan. However, access to more detailed information, such as pharmacy participation, medication costs and the like which are often specific to a particular prescription benefit plan, will be restricted to only those visitors who are enrolled in the subscribing plans.

The system of the invention is best understood by two examples. In the first, the patient is the exemplary female hypertensive. She is enrolled in a plan which subscribes to the system of this invention so she is entitled to access to the Internet site and patient information database from her personal computer. On her first visit she will be requested to fill out a detailed personal profile, including her location information, identification of the particular prescription benefit plan to which she belongs, and her medical and medication history. Once this information is entered into the system, she may use the system over the Internet at her convenience to have prescriptions filled initially and refilled to the extent of her physician's instructions. She can also, as discussed above, use the system to research not only her prescribed medications but also other medications which she considered applicable to her health. In this example her physician has prescribed the branded medication Prinzide® 25 (25 mg of hydrochlorothiazide and 20 mg of lisinopril) to treat her hypertension. It is assumed for this example that she initially has heard from a friend that there may be a generic alternative to her branded medication, but she does not know the identity of that generic alternative. Since the present invention allows a patient to access all of the relevant medication information by entering either the brand name of a proprietary medication or the name of a generic medication, with the quantity prescribed, she is able to bring up a screen on her computer monitor of the type exemplified in Table 1 on the next page.

TABLE 1
Example of Information Provided to Patient:
Simulation of System Response to Patient
Inquiry Regarding Hypertensive Medications
Search Results (within 20 miles of
[designated ZIP Code])

| | | | |
|---|---|---|---|
| | | | 90-Day Supply |
| Pharmacy | 30-Day Supply | 90-Day Supply | By Mail Order |

LISINOPRIL-HCTZ (generic) [view all pharmacies]

| Pharmacy A | Your Price | ◯ | $2.56 | ● | $0.00 ($0.00/mo.) | N/A | |
|---|---|---|---|---|---|---|---|
| Local Address | Plan Pays | | $18.64 | | $55.92 | | |
| Pharmacy B | Your Price | ◯ | $3.85 | ◯ | $2.35 ($0.78/mo.) | N/A | |
| Local Address | Plan Pays | | $18.64 | | $55.92 | | |
| Pharmacy C | Your Price | ◯ | $5.27 | ◯ | $4.10 ($1.36/mo.) | N/A | |
| Local Address | Plan Pays | | $18.64 | | $55.92 | | |
| Pharmacy D | Your Price | N/A | | N/A | | ◯ | $1.18 |
| Mailing Address | Plan Pays | | | | | | $87.10 |

PRINZIDE (Brand) [view all pharmacies]

| Pharmacy A | Your Price | ◯ | $13.92 | ◯ | $23.45 ($7.82/mo.) | N/A | |
|---|---|---|---|---|---|---|---|
| Local Address | Plan Pays | | $18.64 | | $55.92 | | |
| Pharmacy B | Your Price | ◯ | $16.20 | ◯ | $26.77 ($8.92/mo.) | N/A | |
| Local Address | Plan Pays | | $18.64 | | $55.92 | | |
| Pharmacy C | Your Price | ◯ | $17.54 | ◯ | $28.10 ($9.37/mo.) | N/A | |
| Local Address | Plan Pays | | $18.64 | | $55.92 | | |
| Pharmacy D | Your Price | N/A | | N/A | | ◯ | $63.58 |
| Mailing Address | Plan Pays | | | | | | $87.10 |

The screen illustrated in Table 1 organizes the information important to the patient—the availability of the medication she needs and the choices she has for different forms, generic or brand name, and pharmacy, and what the comparative costs of her choices to her will be. It will be observed that the information includes both the generic combination medication hydrochlorothiazide/lisinopril, which may be produced by several different pharmaceutical manufacturers, and a branded version “Prinzide®” which is proprietary to a specific manufacturer. It will be understood that these two are exemplary only, and where numerous manufacturers provide branded versions of a medication the system is capable of displaying as many as are applicable for patients, payers and plans. Similarly, where there are several different forms and/or dosages available, the system is capable of displaying as many as are applicable. Common limitations in the items displayed might be, for instance, that a plan will allow for a medication in tablet form but not in liquid form, or allow for a direct release tablet form but not an extended release form, and so forth. In the present example, the patient's prescription benefit plan for hydrochlorothiazide®/lisinopril pays a flat rate of $18.64 to a retail pharmacy for a 30-day supply and $55.92 for a 90-day supply, regardless of whether the medication is in generic or branded form, as indicated on the “Plan Pays” lines in Table 1. The various retail pharmacies offer the medication at a variety of retail prices. These prices are reflected in the “Your Price” lines in Table 1. The “Your Price” retail cost to the patient is the pharmacy's selling price less the plan payment amount. Thus in the first listing in Table 1 (Pharmacy A; generic hydrochlorothiazide/lisinopril, 30-day supply) the pharmacy's selling price is $21.20. As the plan pays $18.64 the net cost to the patient is $2.56. It will be seen that some pharmacies, especially mail-order pharmacies, do not dispense all forms or quantities of each medication, as indicated by the “N/A” (“not available”) notations in Table 1. This is a common industry practice. Also, Table 1 indicates that the plan will pay more ($87.10) to a 90-day mail order pharmacy to cover not only the drug costs but also the costs of order handling and shipping the medications to the patients, which encourages use of mail order by the patients, since the mail order pharmacies can thus afford to cover their added shipping and handling expenses and still offer prices competitive to the retail pharmacies (as indicated by Pharmacy D's advertised price to the patient for the generic product which is greater than Pharmacy A's price but still less than the prices of Pharmacies B and C for the same product).

From this information the patient can easily make her selection. If she wishes to obtain a branded product, she is free to do so, but she must pay a larger retail price than if she had chosen a therapeutically equivalent but less expensive drug (whether or not the latter is a branded or generic drug). Thus the system allows freedom of choice for patients, but also encourages them to keep health costs down by using less expensive equivalent medications where possible, by making the patient shoulder a greater cost if a higher-priced product is chosen. As illustrated in Table 1, generic drugs of a given type normally have substantially lower costs than do their equivalent branded counterparts, but the price differences between the two groups will be dependent on what particular type of drug of being considered. It will also be observed that in this example one pharmacy (Pharmacy A; generic hydrochlorothiazide/lisinopril; 90-day supply) has a selling price that is no greater than $55.92, the plan's scheduled payment, so that there is no net cost to the patient (i.e., $0.00). This also enhances the efforts of the plan to keep costs down by encouraging patients to make fewer purchases but purchase in larger quantities. In the example in Table 1 the patient has selected this option, as indicated by the black dot [●] signifying her having clicked on the activation icon ( the “◯” in each cost cell) for the $0.00 quantity and pharmacy. Thus the patient benefits by getting her medication at no net cost to her, Pharmacy A benefits by having made a sale to a customer, and the plan benefits by having its member/patient purchase the medication in the generic form and 90-day supply that the plan wishes to encourage.

In the second example, the exemplary adult male patient with hyperlipidemia will in the same manner have been enrolled in a prescription plan whose payer contracts with a PBM who uses the present invention, and the patient will have entered the required personal identification information and health history into the system of the invention. He has also previously obtained medications through the system and has identified a pharmacy and its address where he normally prefers to obtain medications under his plan. He wishes to determine whether there are lower cost medications which are therapeutically equivalent to the branded Zocor® simvastatin that he has been prescribed. He enters the system online and enters “Zocor” as the search term. The system responds with a list of the various dosage levels of Zocor® simvastatin that are available and identifies the medication as a branded product, and provides the on-screen means to select the dosage level (20 mg) and days' supply (90 days) that he wants to price. The system presents a listing of costs as illustrated in Table 2 below for the selected Zocor® simvastatin and for several branded and generic equivalents.

TABLE 2
Example of Information Provided to Patient:
Simulation of System Response to Patient
Inquiry Regarding Antihyperlipidemic Medications
Your Preferred Pharmacy: Pharmacy E [change]

| Select | Drug Name | Price |
|---|---|---|
| ● | Lovastatin 40 mg | $0.00 |
| ◯ | Lescol XL 80 mg | $151.61 |
| ◯ | Lipitor 10 mg | $178.94 |
| ◯ | Pravachol 40 mg | $317.59 |
| ◯ | Zocor 20 mg | $351.77 |

It will be recognized that lovastatin is available in generic form as well as in branded form, while the others are branded products. The “price” listed is the net price to the patient as a member of the particular plan at the patient's preferred pharmacy, based on the difference between what the PBM pays under the plan to a pharmacy for each of the listed medications and what the pharmacy charges as its retail price for each medication. As in the previous example, the PBM's payment for the generic medication is equivalent to or greater than the pharmacy's retail price so the net cost to the patient is $0.00. Having learned of the available alternative of lovastatin, the patient consults with his physician, who approves of his changing from branded Zocor® simvastatin at 20 mg to the generic lovastatin at 40 mg. Table 2 in this example illustrates that with the physician's approval, the patient has selected this alternative, as indicated by the black dot [●] signifying his having reentered the system and clicked on the activation icon (the “◯” in the “Select” column for each medication) for the 90-day supply of generic lovastatin at his net cost of $0.00 at that pharmacy. The savings to the patient thus amounts to $351.77, the difference between what would have been his cost of the branded product Zocor® simvastatin and the generic lovastatin.

As illustrated in Table 1, the system of the invention would have also permitted the patient, had he so chosen, to compare prices for the generic lovastatin at other pharmacies, both retail pharmacies in his locale and mail order pharmacies, to see if the lovastatin is available at a lower price at another pharmacy that he considered sufficiently convenient for him to deal with as an alternative to his current preference, Pharmacy E. The patient can designate a maximum distance for his travel, and the system will list pharmacies within that range, their addresses, distance and price, and the patient can select one of those pharmacies as an alternative to his usually preferred pharmacy, by clicking on the “[change]” icon on the screen simulated in Table 2 and indicating his alternative selection. In the example of Table 2, the patient does not choose to do that, since his net cost is already $0.00 at his preferred pharmacy.

As illustration of yet another aspect of the invention of benefit to patients, the exemplary male patient may learn from the pharmacist upon presenting his Zocor® simvastatin prescription at the pharmacy that there is a generic equivalent medication, lovastatin, which is available at a significantly lower cost. The patient can then enter “lovastatin” into the system to see if his plan covers this medication as an alternative to Zocor® simvastatin and if so, what his comparative net prices would be for each medication. Then having verified that his plan does allow a patient to obtain either, albeit at different costs, he can consult with his physician as described above and, with the physician's approval, switch to obtaining lovastatin as his antilipemic medication. The system therefore not only itself identifies for patients what therapeutically equivalent alternative medications there are to the medication they have been prescribed (as illustrated in both examples) but it also permits patents who learn of possible alternative medications through other sources (for instance, the female patient's friend in the first example and the male patient's pharmacist in the second example) quickly and thoroughly to determine whether such alternative is available under their prescription plan, and if so, at what pharmacies and costs.

It will be understood that in these examples, since they illustrate situations in which the patient is initially not aware of the existence or identity of the lower-priced generic alternative equivalent medication, implementing the transaction may require that before the patient can select the generic drug on-line through the system of this invention, he/she would have had to consult with his/her physician to get the physician's permission to switch from the currently prescribed branded medication to the generic equivalent medication. As noted herein, the method of this invention is not a method for a patient to self-prescribe his/her medications. The terms of the prescription plan will determine how a patient must interact with his/her physician to define the forms, dosages, quantities and refills of the drugs that the patient can obtain though the system. If the physician has prescribed a branded drug but has also indicated that the prescription can be filled with a therapeutically equivalent generic drug, the patient will normally be able to select a lower-cost generic drug on-line without further confirmation of the physician's approval. However, where the physician has not so indicated on the original prescription, the system will still show the generic equivalents and their costs upon the patient's inquiry, but will not let the patient select a generic equivalent for purchase. This ensures that both the patient and the physician are involved in determining the proper course of medication for the patient. The patient can learn of the equivalent generic drug through the system, but must consult With the physician before making a change from the prescribed medication.

It will be noted that nothing in the system of this invention requires that a patient purchase only a specific quantity or form of a medication, or that a pharmacy carry only a specific quantity or form of a medication. Each pharmacy and plan is free to define for its members and customers those quantities and forms which it wishes to carry or support, and not include in its subscription to the plan those quantities and forms which it is not interested in. Of course, many proprietary medications are presently available from only one manufacturer, or are not presently available in generic form, or some medications are available only under medically-determined dosage or quantity limitations or for treatment only of specific conditions. These limitations can also be accommodated by the system of the invention, and while there is a resultant limitation of the patient's choices, those are a function of the medication itself and not of the system.

Once the patient has made a choice by clicking on the appropriate selection icon, the software implementing the invention provides a conventional “shopping cart” order cumulation and payment specification procedure common to many on-line product purchasing systems. This will allow the patient to order several prescription medications at the same time and to indicate the manner in which the patient wishes to pay the net costs due for the selected medications. Once the patients “shopping cart” orders and selected payment method are approved by the system, the system transmits those orders to the pharmacy selected, and filling of the prescription(s) is scheduled and acknowledged. Such acknowledgement and schedule may be communicated to the patient through the system or directly from the pharmacy to email address provided by the patient in his/her original system registration information. Similarly, delivery of the prescription medication will normally be by mail or courier from mail-order pharmacies and by the patient's personal appearance at the pharmacy for local retail pharmacies. However, the system can also accommodate other types of delivery provisions as may be available from individual pharmacies, such as where a local retail pharmacy will offer delivery to shut-in patients or to customers generally within a defined local geographical area.

Additionally, the method of this invention will preferably also identify for the patient cumulative financial data about his or her previous usage of the system, usually on an annual basis. Thus it is contemplated that the system will show on-screen the total dollar value of benefits used so far during the calendar year by the patient as well as the total net amounts the patient has personally paid for the medications obtained under the plan during the year. These data can be implanted to be updated simultaneously with the patient's current access to the system and ordering of new or refill prescription medications. For instance, for the exemplary female hypertensive in the first example assume that she has previously during the year ordered medications totaling $287.50 in value but for which she has had only a total net cost of $60.00 because of the companion benefit payments paid by her prescription plan. If she now selects the net-zero cost generic hydrochlorothiazide/lisinopril medication in the 90-day quantity from Pharmacy A as indicated in Table 1 above, her total value usage will increase to $343.42 but her cumulative net cost will remain at $60.00. If, however, she elects to purchase that same medication and quantity from Pharmacy C (perhaps because Pharmacy C is more conveniently located) her total value usage will still increase to $343.42 but now her cumulative net cost will increase to $65.27. The changes can be shown in real time on the Internet web site screen to that the patient can readily see how her selection of a pharmacy affects her overall payment and plan value history.

Many other features which are of value to patients, other participants and/or the patients' physicians, caregivers and other medical advisors may be incorporated into the method of this invention. For instance, one of significant importance is an ability of the system to identify from the medical and medication history which the patient originally put into the system and/or which has developed as the patient has utilized the invention, when there is a potential for two or more of the medications when present in combination to produce adverse effects or contraindications for the patient. For instance, in the case of the exemplary female hypertensive patient, thiazides in general (including hydrochlorothiazide) are contraindicated for use by patients taking digitalis glycosides, which are commonly used to treat certain cardiac conditions, or patients with impaired hepatic function or renal disease, since the thiazides can cause hypokalemia, hyponatremia and/or hypochloremia. Thus if she should develop a cardiac condition in addition to her hypertension and her physician should wish to prescribe digitalis glycosides, the system of this invention preferably will include means to recognize and advise when she enters that digitalis glycosides prescription into the system, that there are potential adverse reactions from the combination of medications, and she can then consult with her physician before she starts taking the beta blocker. Similarly, if the exemplary female hypertensive subsequently develops a renal condition, upon her updating her medical history in the system, the system will preferable also include means to advise that she should promptly consult her physician about what precautions should be taken with respect to continuing her regimen of hydrochlorothiazide medication.

Yet another desirable feature is to include in the database general information about specific medications and medical conditions and about various types of medications and personal health in general. This will permit members of the public to research information about medications, medical conditions and aspect of health which are of interest to them, and which will enhance their knowledge when consulting with physicians, pharmacists and other health care professionals. It is contemplated that this information will be available without retail pricing or prescription plan payment schedule information, so that this portion of the database may be made available to the general public and not limited only to members of subscribing prescription benefit plans. Thus the exemplary patients will be able to research in depth their respective hypertension and hyperlipidemia as medical conditions and the respective medications hydrochlorothiazide, lovastatin and their analogs, so that their understanding of their health and the medications prescribed for them will be enhanced, and they can more knowledgeably and confidently follow their treatment regimens and discuss those with their physicians. Similarly, someone who is a caregiver for a patient with a particular medical condition can do research on that condition, so that the caregiver can more effectively provide appropriate care to the patient. Numerous other situations in which the ability to research medications or medical conditions will be of general public value and specific value to individuals will be readily evident to those skilled in the art.

Yet another desireable optional feature is for the software implementing the method of the invention to include the capability to compile and organize the data from filed prescriptions in to correlations meaningful to the pharmacies, the PBMs and the prescription benefit plan payers, to allow them to analyze the extent to which the system is producing results positive from their points of view. The data used are aggregated and de-identified. Overall a key goal of prescription benefit plans is to provide prescription medications at a reasonable cost. To achieve this goal the PBMs want to encourage their beneficiaries, the employees and members of the payer organizations, to make wise choices when obtaining prescription medications under their various prescription benefit plans. The PBMs therefore set payment benefit amounts at levels that they believe are reflective of the proper retail price of a medication, and usually, where possible, that is based on the generic form of the medication. Under the current invention, it is possible to track all sales of medications provided under a particular prescription benefit plan, the net price paid by each patient and the pharmacy from which the purchase was made. By analyzing the cumulative purchase data, the PBM can determine the extent to which the plan members have been buying the lower- or lowest-priced versions of the medication and the distribution of patronage of the various pharmacies providing the medication. From these data the PBM can determine where and at what price sales are being made, and whether these sales represent patients focusing on the target products that the PBM wishes to encourage. If not, the PBM can then adjust its payment policies to encourage the participating pharmacies to revise their pricing to entice more patients to focus on those target products.

Similarly, pharmacies can also use such data to their advantage, but analyzing where the various patient purchases are being made and at what price. Each pharmacy can thus tell the extent to which it is competitive in attracting and retaining patients as customers, and perhaps then change its operations and/or pricing to improve its attraction to such customers.

Yet another feature which it is contemplated can be incorporated into the method of this invention is the ability to compile and report information on various hospitals and critical care facilities, such as treatment outcomes, costs and issues involving medication safety. Such is more likely to involve obtaining basic data from hospital or hospital-review sources, such as governmental data compilations and reports, but these can be entered into the system and the data and various analyses of them can be published such as in tabular form from the database.

The implementation of this method by computer software and publication and interactive usage through an Internet web site is based on conventional and well-known computer and software technology. Such implementation will be well understood by those skilled in the art.

Although several embodiments of the invention have has been described above by way of example only, it will be understood by those skilled in the field that numerous variations and modifications may be made to the disclosed embodiments without departing from the scope or spirit of the invention, as it is defined by the appended claims.

We claim:

1. A method for providing choice-enhancing prescription medication cost and availability information to prescription medication patients, which method comprises:
providing a database on an electronic network, which database comprises dosage and form data on a plurality of therapeutically or clinically equivalent medications and price and location data on a plurality of pharmacies dispensing said medications;
compiling in conjunction with said database payment benefit data from at least one prescription benefit plan subscribing to said database, said payment benefit data comprising a payment amount said payment benefit plan will pay to said pharmacies on behalf of a member of said payment benefit plan for said medication when said medication is dispensed by a said pharmacy to said member in accordance with a prescription written for said member;
permitting an individual member of said prescription benefit plan access to said database and enabling said member to enter personal location, medical and prescription information into a personal medical history in conjunction with said database; and
upon accessing of said database by said member, correlating said database data with said payment benefit data and said medical history into a location-, plan- and medication-specific compilation of prescription cost and availability information and presenting said compilation via said electronic network to said member;
whereby said member is informed of a plurality of therapeutically or clinically equivalent medication choices and pharmacies conveniently located to said member or available to said member by mail, which dispense said medication for which said member has a prescription, and is also informed of the net cost of said medication to said member at each said pharmacy under said member's prescription benefit plan, such that said member's ability to freely select which pharmacy to patronize based on said member's personal criteria is enhanced.

2. A method as in claim 1 further comprising means for said member to specific method of net payment for said medication and an order for said medication to be transmitted to said pharmacy for fulfillment.

3. A method as in claim 2 further comprising means for acknowledgement of said order to be communicated to said member.

4. A method as in claim 2 further comprising means for communication to said member of a schedule for fulfillment of said order and a manner in which said filled order may be provided to said member.

5. A method as in claim 1 further comprising means for communicating to said member real time data showing cumulative value of prior prescription benefits obtained and cumulative prior net payments made.

6. A method as in claim 1 wherein said database further comprises data regarding at least one combination of medications and adverse reactions or contraindications which a user may experience or cautions which said user should observe if said combination of medications is taken.

7. A method as in claim 1 wherein said database further comprises data generally describing properties, symptoms or physiological effects of individual medications, diseases or health conditions.

8. A method as in claim 1 wherein said that portion of said database which comprises medical, health and medication data not identified by financial elements, prescription benefit plan or personal user data is made available via said electronic network to persons generally and not limited solely to access by said prescription benefit plan members.

9. A method as in claim 1 further comprising means for compiling system usage and pharmacy selection data in form from which subscribing prescription benefit plans and pharmacies may analyze system performance and user utilization patterns, whereby any such prescription benefit plan or pharmacy may assess its own operations in relation to its participation in said method.

10. A method as in claim 1 further incorporating location-specific data on hospital services, which data are relevant to users' selection of hospital care.

11. A method as in claim 10 wherein said data include information on treatment outcome, costs and medication safety.
